(19) United States
(12) Patent Application Publication
(10) Pub. No.: US 2023/0338151 A1
(43) Pub. Date: Oct. 26, 2023

(54) AUGMENTATION DEVICE AND METHOD FOR ADAPTING AN AUGMENTATION DEVICE

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/303,291

(22) Filed: Apr. 19, 2023

(30) Foreign Application Priority Data
Apr. 25, 2022 (EP) 22169675.0

(51) Int. Cl. A61F 2/30

(52) U.S. Cl. CPC A61F 2/30734; A61F 2002/30215; A61F 2002/30217; A61F 2002/30736; A61F 2002/3069; A61F 2002/30822; A61F 2002/30828; A61F 2002/30561; A61F 2002/30677

(57) ABSTRACT

The invention relates to an augmentation device comprising an annular cone surrounding a channel which extends through the cone from a proximal cone end to a distal cone end of said cone. The invention furthermore relates to a method for adapting a cone size of such an augmentation device.

### CROSS-REFERENCE TO RELATED APPLICATIONS

This application claims priority pursuant to 35 U.S.C. 119 to European Application No. 22169675.0, filed Apr. 25, 2022, which application is incorporated herein by reference in its entirety.

### FIELD

The invention relates to an augmentation device comprising an annular cone which surrounds a channel which extends through the cone from a proximal cone end to a distal cone end of said cone.

The invention furthermore relates to a method for adapting a cone size of such an augmentation device.

The subject matter of the invention is in particular an augmentation device for use in joint endoprosthesis operations, in particular revision joint endoprosthesis operations.

The augmentation device according to the invention is suitable for strengthening or even partially replacing debrided bone tissue and thus allowing a joint endoprosthesis—in particular, a revision endoprosthesis—to be anchored securely and stably in a bone canal. In addition, the augmentation device according to the invention serves in particular to uniformly introduce force into the surrounding bone tissue in the course of implantation of a joint endoprosthesis, in particular a revision joint endoprosthesis.

### BACKGROUND OF THE INVENTION

In orthopedic surgery, septic revisions of joint endoprostheses infected with microorganisms must unfortunately be carried out to a certain extent. The infected joint endoprostheses are thereby explanted and the infected or necrotic tissue is removed. This removal of infected/necrotic tissue is referred to as debridement. In this case, a substantial loss of bone tissue can occur in the region of the removed joint endoprosthesis—in particular in the case of previously damaged bone tissue substance, e.g., due to osteoporosis—which can lead to problems when anchoring a revision joint endoprosthesis. In order to strengthen or partially replace the remaining bone tissue and to ensure as uniform an introduction of force as possible into the remaining bone tissue during use of the revision joint endoprosthesis, one possible treatment option is the use of an augmentation device in the form of a metal cone. To this end, the infected bone tissue is removed, and the resulting cavity is filled by inserting the augmentation device. The conical design of the augmentation device serves to receive a stem of a joint prosthesis or a revision joint endoprosthesis in a centrally extending channel of the augmentation device and to anchor it there, for example using bone cement. The augmentation device itself is designed such that it can be inserted into the cavity formed by debridement. Such an augmentation device is described, for example, in the patent specification U.S. Pat. No. 8,506,645 B2.

Usually, the augmentation devices available on the market have a defined, predetermined size and cannot be adapted to the respective anatomical condition of the patient. Therefore, augmentation devices of different sizes are offered on the market.

In the patent specification EP 2 319 558 B1, an augmentation device is described whose size, in particular whose outer diameter, can be varied within certain limits. For this purpose, the augmentation device comprises an annular cone which comprises at least one axially extending flexural joint. The at least one joint allows the cone to be compressed by external pressure, which reduces the outer diameter of the augmentation device.

When using joints, in particular bending joints, it is considered disadvantageous that the adjustment of the size, in particular of the outer diameter, of the augmentation device is accompanied by a marked deformation of the augmentation device. Due to the adjustment of the size, the augmentation device has a “bend” which makes it difficult to fit the augmentation device into the bone of the patient and has a disadvantageous effect on the implantation of the joint endoprosthesis or the revision joint endoprosthesis. In addition, an adaptation of the size of the augmentation device is possible only to a small extent. An adaptation of the axial extent of the augmentation device is not possible in the course of a running operation in an operating room.

In patent application US 2018/0271658 A1, an augmentation device made of a mesh made of titanium is described. By cutting away parts of the fabric, the augmentation device can be adapted to the anatomy of a patient.

A disadvantage of an augmentation device made of a metal mesh is that it is not dimensionally stable, which both negatively affects the stability of a prosthesis implanted therewith. In addition, the force required upon implantation of the prosthesis, which force acts on the augmentation device and the tissue of the patient connected to it, cannot be transmitted uniformly. In particular, the latter can lead to damage to the patient's bone tissue. In addition, the cutting away can lead to sharp-edged mesh strands which represent a risk of injury to the surgeon and the patient.

An augmentation device is therefore desirable which is dimensionally stable and can be simply and quickly adapted in terms of its size, in particular its diameter and its length. The adaptation of the size should be possible without deformation of the augmentation device. Furthermore, the adaptation of the size should be implementable with means which are typically used in the course of an orthopedic operation, such as saws, for example.

### OBJECTS

It is an object of the present invention to at least partially overcome one or more of the disadvantages resulting from the prior art.

In particular, an augmentation device is to be provided which is dimensionally stable and can be adapted simply and quickly, in terms of its size, to the anatomical conditions of a patient. Furthermore, the augmentation device should not be deformed when adapting the size. Furthermore, the augmentation device should not represent a risk of injury after adapting the size.

Another aim of the invention is to provide a method with which an augmentation device can be adapted in terms of its size and which avoids limitations of conventional implantation methods.

### PREFERRED EMBODIMENTS OF THE INVENTION

The features of the independent claims contribute to at least partially fulfilling at least one of the aforementioned objects. The dependent claims provide preferred embodiments which contribute to at least partially fulfilling at least one of the objects.

A first embodiment of the invention is an augmentation device comprising an annular cone which surrounds a channel which extends through the cone from a proximal cone end to a distal cone end of said cone, wherein the cone consists by at least 50 percent by volume, preferably at least 70 percent by volume, more preferably at least 90 percent by volume, based on the total volume of the cone, of a biocompatible polymer, and is subdivided into annular cone segments by at least three, preferably three to eight, more preferably three to six, more preferably three to five radially circumferential grooves in a lateral cone surface opposite the channel,

wherein the grooves have a groove depth of at least 1 mm and a groove width of at least 1 mm, and form sawing guides in order to adapt a cone size of the cone by separating one or more cone segments.

In one embodiment of the augmentation device, the lateral cone surface is roughened or porous, at least in portions. This embodiment is a second embodiment of the invention, which is preferably dependent upon the first embodiment of the invention.

In one embodiment of the augmentation device, the lateral cone surface is formed by at least 70 percent by area, preferably at least 80 percent by area, more preferably at least 90 percent by area, based the entire lateral cone surface, of tantalum, a tantalum alloy, titanium, a titanium alloy, or stainless steel. This embodiment is a third embodiment of the invention which is preferably dependent on the first or second embodiment of the invention.

In one embodiment of the augmentation device, the biocompatible polymer is a PMMA bone cement. This embodiment is a fourth embodiment of the invention which is preferably dependent upon one of the preceding embodiments of the invention.

In one embodiment of the augmentation device, the PMMA bone cement contains at least one antibiotic. This embodiment is a fifth embodiment of the invention which is preferably dependent upon the fourth embodiment of the invention.

In one embodiment of the augmentation device, the cone has radially circumferential inner grooves in a cone inner surface facing toward the channel, which inner grooves are arranged opposite the grooves, in particular radially opposite. This embodiment is a sixth embodiment of the invention which is preferably dependent upon one of the preceding embodiments of the invention.

In one embodiment of the augmentation device, the lateral cone surface has at least two axially extending axial grooves with an axial groove depth of at least 1 mm and an axial groove width of at least 1 mm, each of which is connected at least to one of the grooves. This embodiment is a seventh embodiment of the invention, which is preferably dependent upon one of the preceding embodiments of the invention.

In one embodiment of the augmentation device, the cone segments are separated from one another in a stepped manner. This embodiment is an eighth embodiment of the invention, which is preferably dependent upon one of the preceding embodiments of the invention.

A ninth embodiment of the invention is a method for adapting a cone size of an augmentation device, in particular an augmentation device according to any one of the preceding embodiments of the invention, comprising a step of separating one or more cone segments at the proximal cone end, at the distal cone end, or at the proximal and at the distal cone end by sawing, cutting, or breaking along the radially circumferential groove or grooves.

One embodiment of the method for adapting the cone size of an augmentation device according to the seventh embodiment of the invention comprises a step of separating a conical part from the cone by sawing, cutting, or breaking along two of the axially extending grooves. This embodiment is a tenth embodiment of the invention which is preferably dependent on the ninth embodiment of the invention.

### GENERAL

In the present description, range specifications also include the values specified as limits. An indication of the type “in the range of X to Y” with respect to a variable A consequently means that A can assume the values X, Y and values between X and Y. Ranges delimited on one side of the type “up to Y” for a variable A accordingly mean, as a value, Y and less than Y.

Some of the described features are linked to the term “substantially.” The term “substantially” is to be understood as meaning that, under real conditions and manufacturing techniques, a mathematically exact interpretation of terms such as “superimposition,” “perpendicular,” “diameter,” or “parallelism” can never be given exactly, but only within certain manufacturing-related error tolerances. For example, “substantially perpendicular axes” enclose an angle of 85 degrees to 95 degrees relative to one another, and “substantially equal volumes” comprise a deviation of up to 5% by volume. An “apparatus consisting substantially of plastic material” comprises, for example, a plastics content of >95 to <100% by weight. A “substantially complete filling of a volume B” comprises, for example, a filling of >95 to <100% by volume of the total volume of B.

The terms “proximal” and “distal” serve only to designate the spatially opposite ends of the augmentation device, of the cone, or of other structural units of the augmentation device and of the augmentation system, and do not permit any conclusions to be drawn about the orientation of the augmentation device implanted in a human body. “Distally to . . . ” and “proximally to . . . ” or similar formulations correspondingly express only the spatial arrangement of two structural units of the augmentation device and the augmentation system in relation to one another.

### DETAILED DESCRIPTION

A first subject matter of the invention relates to an augmentation device comprising an annular cone which surrounds a channel which extends through the cone from a proximal cone end to a distal cone end of said cone, wherein the cone consists by at least 50 percent by volume, preferably at least 70 percent by volume, more preferably at least 90 percent by volume, based on the total volume of the cone, of a biocompatible polymer, and is divided into annular cone segments by at least three, preferably three to eight, more preferably three to six, more preferably three to five radially circumferential grooves in a lateral cone surface opposite the channel,

wherein the grooves have a groove depth of at least 1 mm and a groove width of at least 1 mm, and form sawing guides in order to adapt a cone size of the cone by separating one or more cone segments.

The augmentation device comprises an annular cone. A cone is a conical, in particular frustoconical, shaped component comprising a proximal cone end and a distal cone end axially opposite the proximal cone end, wherein a cone outer diameter decreases from one cone end to the other cone end. The cone according to the invention preferably tapers from the proximal cone end in the direction of the distal cone end.

The cone is annular, or, in other words, tubular. The cone thus encloses a channel which extends axially through the cone from the proximal cone end to the distal cone end. The channel is formed by an inner cone surface facing toward the channel. The term “annular” comprises, in an axial plan view of the cone, bodies comprising a circular, elliptical, angular, e.g., quadrangular, pentagonal, or hexagonal, and irregular circumference, wherein bodies comprising an elliptical circumference are preferred due to better implantability in a patient, in particular in a bone canal of a patient.

The channel serves to receive and fix a stem of a joint endoprosthesis or of a revision joint endoprosthesis. For this purpose, the stem of the corresponding endoprosthesis can be inserted into the channel, in particular from the proximal cone end. The channel comprises a channel diameter which is determined by a cone inner diameter. In one embodiment, the cone inner diameter can be configured to be constant over the entire axial extent of the channel or of the cone. In a further embodiment, the cone inner diameter decreases uniformly to the cone outer diameter from the proximal cone end in the direction of the distal cone end. In this embodiment, the cone has a conical wall thickness that is substantially equal over the entire axial extent of the cone.

The cone wall thickness can, for example, be in a range of 2 to 30 mm, preferably 2 to 15 mm, more preferably 2 to 10 mm.

The axial extent of the cone can, for example, be in a range from 30 mm to 50 mm, so that the augmentation device is suitable or adaptable for an optimally wide bandwidth of different anatomical conditions of patients.

The cone has a lateral cone surface opposite the channel, which surface is thus external. In the lateral cone surface, at least three, preferably three to eight, more preferably three to six, more preferably three to five, radially circumferential grooves run which divide the cone into annular cone segments.

Grooves are elongated depressions in the lateral cone surface. The grooves are radially circumferential and preferably extend substantially in a respective plane which is respectively preferably substantially perpendicular to a longitudinal axis of the augmentation device. The grooves thus run substantially “horizontally” with respect to the longitudinal axis of the augmentation device.

The cone wall thickness is reduced in the region of the grooves, so that the cone is subdivided by the grooves into annular cone segments. For example, three grooves subdivide the cone into four cone segments, and four grooves subdivide the cone into five cone segments.

The grooves facilitate the separation of individual cone segments so that the augmentation device has an adaptable cone size, in particular an adaptable maximum and/or minimum cone outer diameter and an adaptable axial extent of the cone.

The grooves have a groove depth of at least 1 mm and a groove width of at least 1 mm, so that the grooves serve as sawing guides for means for the separation of individual cone segments, for example by sawing, cutting, or breaking. Suitable means for separating are, for example, saws, in particular circular saws; tongs; and shears. For example, breaking edges can serve for the breaking of cone segments.

In order not to negatively affect the structural stability of the cone, it is preferred that the groove depth corresponds to not more than one quarter of the cone wall thickness.

In order to represent a good sawing guide for means for separating individual cone segments, it is preferred that the groove width corresponds to not more than 5 mm, preferably not more than 4 mm, more preferably not more than 3 mm.

The grooves thereby show suitable locations for separating individual cone segments and, in particular, facilitate a controlled and safe separation of individual cone segments along the corresponding groove or grooves. In particular upon sawing and cutting of individual cone segments, the grooves reduce risk of injury to the surgeon, for example due to a reduced danger of a saw slipping on the lateral surface of the cone. Moreover, the grooves can be used as predetermined breaking points for the controlled breaking of individual cone segments.

In one embodiment, the grooves respectively extend around at least 90% of the respective cone outer diameter. In a further embodiment, the grooves respectively extend around at least 95% of the respective cone outer diameter. The grooves preferably respectively circumscribe the entire respective cone outer diameter of the cone. The latter corresponds to grooves in the form of a closed “ring”.

Preferably, the grooves are distributed substantially uniformly over the lateral surface, so that the individual cone segments comprise a substantially identical axial extent.

In order to be able to adapt the augmentation device optimally well to the anatomical conditions of a patient, the cone segment can comprise different axial extents. For example, the grooves are arranged such that the cone segments comprise an axial extent in the range of from 1 mm to 10 mm, preferably in the range of from 2 mm to 8 mm.

Preferably, the cone is designed to be solid, so that the cone inner surface and the cone lateral surface are not connected, in terms of fluid conduction to one another through the cone, for example via a feedthrough or a conduit. A solid cone improves the structural integrity of the augmentation device and thus allows an improved introduction of force in the course of an operation.

Based on the total volume of the cone, the cone consists by at least 50 percent by volume, preferably at least 70 percent by volume, more preferably at least 90 percent by volume, of a biocompatible polymer. Preferably, the cone inner surface is formed entirely from the biocompatible polymer. In one embodiment, the cone consists essentially entirely of a biocompatible polymer. An advantage of a biocompatible polymer is that it allows a rapid adaptation of the cone size by separating individual cone segments, and at the same time gives the cone a structural stability, and thus dimensional stability, which enables a uniform force transmission upon implantation of the augmentation device and a corresponding prosthesis. In addition, in the case of a biocompatible polymer, only edges which represent no or only a slight risk of injury to the surgeon result after the separation of individual cone segments. Furthermore, a biocompatible polymer can be machined with means which are accessible anyway during a surgical operation—for example saws, in particular bone saws, or shears—in order to adapt the conical size.

Examples of biocompatible polymers are PEEK, PES, polyamides, and polyimides, with polyamide-12 being preferred in the case of the polyamides.

One embodiment of the augmentation device is characterized in that the biocompatible polymer is a PMMA bone cement. Since the fastening of a prosthesis in the channel of the augmentation device usually takes place by means of a PMMA bone cement, a cone likewise made of a PMMA bone cement offers the greatest possible compatibility. In order to increase the compatibility with the PMMA bone cement used for fastening a prosthesis, the cone inner surface is preferably formed substantially completely from a PMMA bone cement.

The PMMA bone cement can be loaded with further materials, such as, for example, fillers or a radiopaque material such as, for example, barium sulfate or zirconium oxide.

One embodiment of the augmentation device is characterized in that the PMMA bone cement contains at least one antibiotic. For example, the PMMA bone cement can contain two, three, or four antibiotics. Examples of antibiotics are gentamicin, tobramycin, amikacin, vancomycin, teicoplanin, ramoplanin, daptomycin, colistin, and meropenem, wherein gentamicin and/or vancomycin is preferred.

Preferably, the PMMA bone cement contains at least one antibiotic, in particular at least gentamicin or vancomycin, and is substantially free of radiopaque material since, due to their structural hardness, radiopaque materials, in particular zirconium oxide, could hinder or impede a separation of individual cone segments, for example by blunting of the saw teeth of a saw upon separation of a cone segment along a groove.

In one embodiment of the augmentation device, the lateral cone surface is designed to be smooth.

One embodiment of the augmentation device is characterized in that the lateral cone surface is, at least in portions, preferably by at least 80 percent by area, more preferably at least 90 percent by area, in each case based on the entire lateral cone surface, more preferably substantially completely roughened or porous, preferably open-porous. In particular, the lateral cone surface between and/or next to the grooves is at least in portions, preferably at least 80 percent by area, more preferably at least 90 percent by area, respectively with respect to the entire lateral cone surface, more preferably substantially completely roughened or porous, preferably open-porous. In one embodiment, the lateral surface is provided with blind holes in order to provide a roughened lateral surface. The blind holes thereby preferably have a blind hole diameter in the range of from 300 μm to 500 μm. A roughened or porous, preferably open-porous, lateral surface improves growth of bone tissue on and/or into the augmentation device, and thus improves the stability of the augmentation device in the implanted state. Such a lateral surface forms spongy bone tissue.

The lateral surface can consist of or comprise the biocompatible polymer or other biocompatible materials such as, for example, biocompatible metals.

One embodiment of the augmentation device is characterized in that the lateral cone surface consists of at least 90 percent by area, preferably at least 95 percent by area, respectively with respect to the entire lateral cone surface, more preferably substantially completely, of tantalum, a tantalum alloy, titanium, a titanium alloy, or stainless steel. The aforementioned materials improve an entry and/or growth of bone tissue on and/or into the augmentation device and thus improve the stability of the augmentation device in the implanted state.

In one embodiment, the lateral surface of tantalum, a tantalum alloy, titanium, a titanium alloy, or stainless steel is provided on the cone made of a biocompatible polymer via a coating method such as, for example, vapor deposition. The layer of tantalum, a tantalum alloy, titanium, a titanium alloy, or stainless steel can thereby a layer thickness in a range of from 10 μm to 1000 μm.

In a further embodiment, the lateral surface of tantalum, a tantalum alloy, titanium, a titanium alloy, or stainless steel is provided as a partial cone which is connected to a further partial cone made of the biocompatible polymer, for example by adhesive bonding, clamping, or welding, in order to provide the augmentation device. The layer of tantalum, a tantalum alloy, titanium, a titanium alloy, or stainless steel can thereby have a layer thickness in a range of from 1 mm to 7 mm.

Preferably, the augmentation device consists of a cone which consists of at least 50 percent by volume, preferably at least 70 percent by volume, more preferably at least 90 percent by volume, of a biocompatible polymer, wherein the bone cement preferably contains at least one antibiotic, and wherein the remaining percent by volume consists of the lateral cone surface consisting of tantalum, a tantalum alloy, titanium, a titanium alloy, or stainless steel.

In one embodiment, the cone inner surface of the cone is shaped smooth.

One embodiment of the augmentation device is characterized in that the cone has radially circumferential inner grooves in the cone inner surface facing toward the channel, which inner grooves are arranged opposite the grooves. Preferably, each groove is arranged opposite, in particular radially opposite, an inner groove. The groove and the inner groove situated opposite it reduce the shell thickness of the cone so that individual cone segments can be separated in a simplified manner from the augmentation device, in particular by sawing, cutting, or breaking.

Inner grooves, just like the grooves, are elongated depressions. While the grooves extend in the lateral cone surface, the inner grooves by contrast extend in the cone inner surface. The inner grooves are radially circumferential and preferably extend substantially in a respective plane which respectively is preferably substantially perpendicular to a longitudinal axis of the augmentation device. The inner grooves thus extend substantially “horizontally” with respect to the longitudinal axis of the augmentation device. The grooves and the inner grooves arranged opposite them respectively preferably extend substantially in a common plane which lies substantially perpendicular to the longitudinal axis of the augmentation device.

Preferably, the inner grooves have an inner groove depth in a range of from 1 mm up to 25% of the wall thickness of the cone, and an inner groove width in a range of from 1 mm to a maximum of 5 mm, preferably not more than 4 mm, more preferably not more than 3 mm. This facilitates separation of individual cone segments for adapting the cone size and achieves a sufficient structural integrity of the augmentation device so that a uniform application of force can take place upon implantation of a prosthesis via the augmentation device.

One embodiment of the augmentation device is characterized in that the lateral cone surface has at least two axially extending axial grooves with an axial groove depth of at least 1 mm and an axial groove width of at least 1 mm, each of which is connected at least to one of the grooves. The axial grooves extend in particular in the direction of the longitudinal axis of the augmentation device.

A separation of an axially extending conical part between two of the axial grooves is simplified by the axial grooves. The separation of a conical part enables an improved adaptation of the augmentation device to the anatomical conditions of a patient. The cone preferably has at least four axial grooves so that, via a separation along the respective second of the axial grooves, at least two disjoint conical parts can be separated in a simplified manner. Preferably, two of the axial grooves respectively have a radial distance from one another in a range of from 2 mm to 5 mm, so that corresponding conical parts with a width in this region can be separated.

The axial grooves have a groove depth of at least 1 mm and an axial groove width of at least 1 mm, so that the axial grooves serve as sawing guides for means for separating a conical part, for example by sawing, cutting, or breaking. Suitable means for separating are, for example, saws, in particular circular saws; tongs; and shears.

In order to not negatively affect the structural stability of the cone, it is preferred that the axial groove depth corresponds to not more than one quarter of the cone wall thickness.

In order to provide a good guide for means for separating a conical part, it is preferred that the axial groove width corresponds to not more than 5 mm, preferably not more than 4 mm, more preferably not more than 3 mm.

The axial grooves thereby indicate to the user suitable points for separating a conical part and, in particular, facilitate a controlled and safe separation of a conical part along the corresponding axial grooves. In particular upon sawing off and cutting off a conical part, a risk of injury to the surgeon is reduced by the axial grooves, for example by reducing a risk that a saw will slip on the cone lateral surface. The axial grooves can also be used as predetermined breaking points for the controlled breaking of a conical part.

In order to separate a conical part from the cone, for example, it is possible to saw along two axial grooves. The axial grooves preferably extend from the proximal cone end in the direction of the distal cone end, so that a simplified separation of a conical part is enabled at least in the region of the proximal cone end.

The axial grooves are connected at least to one of the grooves. For example, the axial grooves can open into a groove or extend axially beyond a groove. Since the axial grooves are connected to at least one groove, a simplified separation of a conical part along two of the axial grooves and along the at least one groove is enabled.

In one embodiment, the axial grooves extend beyond one of the grooves and are connected to one another, at least at their respective distal axial groove ends, via a connecting groove. For example, the axial grooves are connected to one another with one or two connecting grooves which extend between at least two of the grooves between the axial grooves. This enables a simplified separation of a conical part which has an axial extent which does not necessarily correspond to a multiple of the axial extent of a cone segment.

In one embodiment, the at least two axial grooves extend over the entire axial extent of the cone, so that a separating of a conical part with an axial extent corresponding to the axial extent of the cone is enabled in a simplified manner by the cone. The resulting cone is present in the form of an open ring after removal of the conical part.

In one embodiment of the augmentation device, the lateral surface is of continuous design, so that the cone outer diameter decreases in the same direction from one cone end to the other cone end, preferably from the proximal cone end to the distal cone end.

One embodiment of the augmentation device is characterized in that the cone segments are separated from one another in a stepped manner. The cone outer diameter thereby decreases in a stepped manner from one of the cone segments to its adjacent cone segment. The grooves preferably run between adjacent step-like cone segments such that, during a separation along the corresponding groove, a substantially complete separation of the two cone segments from one another is made possible.

A further subject matter of the invention relates to a method for adapting a cone size of an augmentation device according to any one of the preceding embodiments, comprising a step of separating one or more cone segments at the proximal cone end, at the distal cone end, or at the proximal cone end and at the distal cone end, by sawing, cutting, or breaking along the radially circumferential groove or grooves.

In order to reduce the cone size in the form of a maximum cone outer diameter, one or more cone segments are separated at the cone end with the larger cone outer diameter; this is preferably the proximal cone end.

In order to increase the cone size in the form of a minimum cone outer diameter, one or more cone segments are separated at the cone end with the smaller cone outer diameter; this is preferably the distal cone end.

In order to adapt both the maximum cone outer diameter and the minimum cone outer diameter, at least one cone segment is separated at both the proximal and the distal cone end.

Each separation of a cone segment reduces the axial extent of the augmentation device.

In order to adapt the augmentation device even better to the anatomical conditions of a patient, one embodiment of the method is characterized in that the method additionally comprises a step of separating a conical part from the cone by sawing, cutting, or breaking along two of the axially extending axial grooves. An augmentation device comprising at least two axial grooves is used for this embodiment. In one embodiment of the method, two conical parts are separated from the cone by sawing, cutting, or breaking along two pairs of axial grooves. The conical parts are preferably arranged at opposite positions, preferably both at the proximal end of the cone. The conical part can be separated before or after the separation of one or more cone segments.

The cone of the augmentation device preferably consists by at least 50 percent by volume, preferably at least 70 percent by volume, more preferably at least 90 percent by volume, based on the total volume of the cone, of a biocompatible polymer in the form of a PMMA bone cement. PMMA bone cements have been used for a long time in medical applications and are based upon the work of Sir Charnley. PMMA bone cements can thereby be produced from a bone cement powder as a first starting component and a monomer liquid as a second starting component. With a suitable composition, the two starting components can be storage-stable, separately from one another. When the two starting components are brought into contact with one another, a plastically-deformable bone cement paste is produced by the swelling of the polymer components of the bone cement powder. In this case, polymerization of the monomer by radicals is initiated. As the polymerization of the monomer progresses, the viscosity of the bone cement paste increases until it cures completely.

Bone cement powder is understood to mean a powder that comprises at least one particulate polymethyl methacrylate and/or a particulate polymethyl methacrylate copolymer. Examples of copolymers are styrene and/or methyl acrylate. In one embodiment, the bone cement powder can additionally comprise a hydrophilic additive which supports the distribution of the monomer liquid within the bone cement powder. In a further embodiment, the bone cement powder can additionally comprise an initiator which initiates the polymerization. In a further embodiment, the bone cement powder can additionally comprise a radiopaque material. In yet another embodiment, the bone cement powder can additionally comprise pharmaceutically-active substances, such as antibiotics.

The bone cement powder preferably comprises, as a hydrophilic additive, at least one particulate polymethyl methacrylate and/or a particulate polymethyl methacrylate copolymer, an initiator, and a radiopaque material, or consists of these components. More preferably, the bone cement powder comprises at least one particulate polymethyl methacrylate and/or a particulate polymethyl methacrylate copolymer, an initiator, a radiopaque material, and a hydrophilic additive, or consists of these components. Most preferably, the bone cement powder comprises at least one particulate polymethyl methacrylate and/or a particulate polymethyl methacrylate copolymer, an initiator, a radiopaque material, a hydrophilic additive, and an antibiotic, or consists of these components.

According to the invention, the particle size of the particulate polymethyl methacrylate and/or of the particulate polymethyl methacrylate copolymer of the bone cement powder can correspond to the sieve fraction of less than 150 μm, preferably less than 100 μm.

According to the invention, the hydrophilic additive can be designed in particulate and/or fibrous form. In a further embodiment, the hydrophilic additive can be slightly soluble, and preferably insoluble, in methyl methacrylate. In a further embodiment, the hydrophilic additive can have an absorption capacity of at least 0.6 g methyl methacrylate per gram of hydrophilic additive. In a further embodiment, the hydrophilic additive can comprise a chemical substance comprising at least one OH group. In this case, the hydrophilic additive can preferably have covalently-bonded OH groups at its surface. Examples of such preferred hydrophilic additives can be additives selected from the group comprising cellulose, oxycellulose, starch, titanium dioxide, and silicon dioxide, wherein pyrogenic silicon dioxide is particularly preferred. In one embodiment, the particle size of the hydrophilic additive can correspond to the sieve fraction of less than 100 μm, preferably less than 50 μm, and most preferably less than 10 μm. The hydrophilic additive can be contained in an amount of 0.1 to 2.5% by weight, based on the total weight of the bone cement powder.

According to the invention, the initiator can contain dibenzoyl peroxide or consist of dibenzoyl peroxide.

According to the invention, a radiopaque material is understood to mean a substance that makes it possible to make the bone cement visible on diagnostic X-ray images. Examples of radiopaque materials can include barium sulfate, zirconium dioxide, and calcium carbonate.

According to the invention, the pharmaceutically-active substance can comprise one or more antibiotics and, optionally, added cofactors for the one or more antibiotics. Preferably, the pharmaceutically-active substance consists of one or more antibiotics and, optionally, added cofactors for the one or more antibiotics. Examples of antibiotics include, inter alia, gentamicin, clindamycin, and vancomycin.

According to the invention, the monomer liquid can comprise the monomer methyl methacrylate or consist of methyl methacrylate. In one embodiment, the monomer liquid comprises, in addition to the monomer, an activator dissolved therein, such as N,N-dimethyl-p-toluidine, or consists of methyl methacrylate and N,N-dimethyl-p-toluidine.

The features disclosed for the augmentation device are also disclosed for the method, and vice-versa.

### BRIEF DESCRIPTION

In the following, the invention is illustrated further, by way of example, by figures. The invention is not limited to the figures.

Shown are:

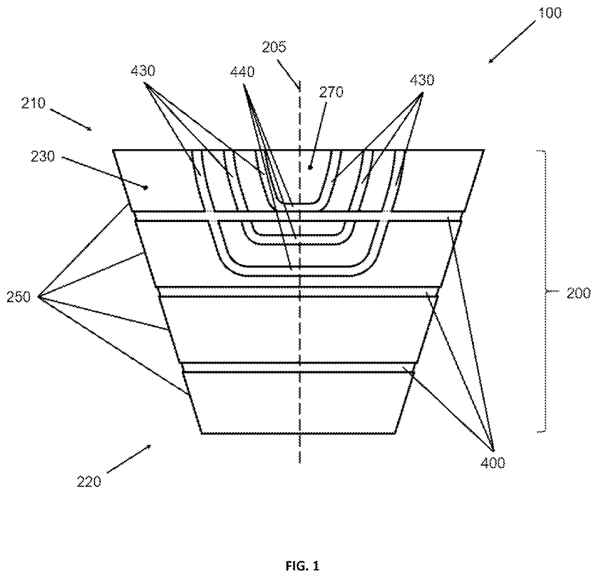

FIG. 1 a schematic side view of an augmentation device,

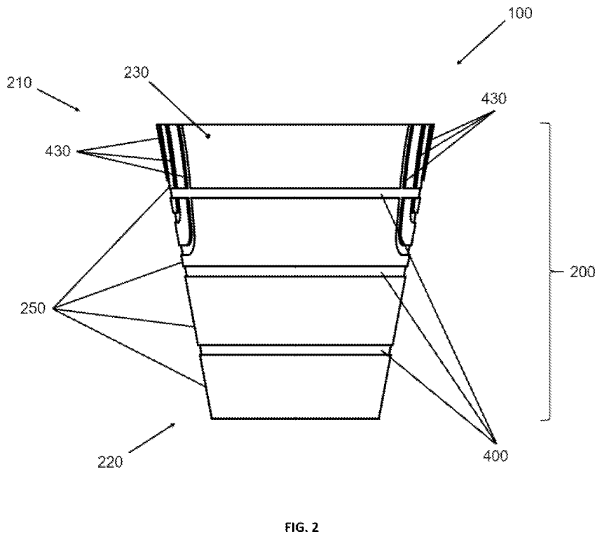

FIG. 2 a further schematic side view of the augmentation device from FIG. 1,

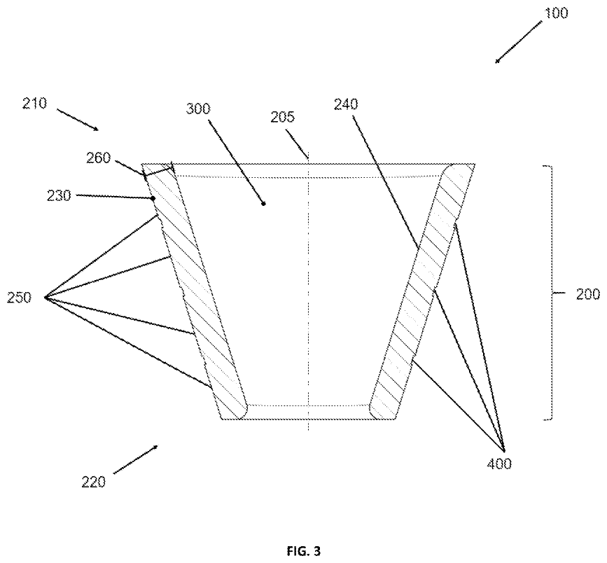

FIG. 3 a schematic longitudinal section of the augmentation device of FIGS. 1 to 3,

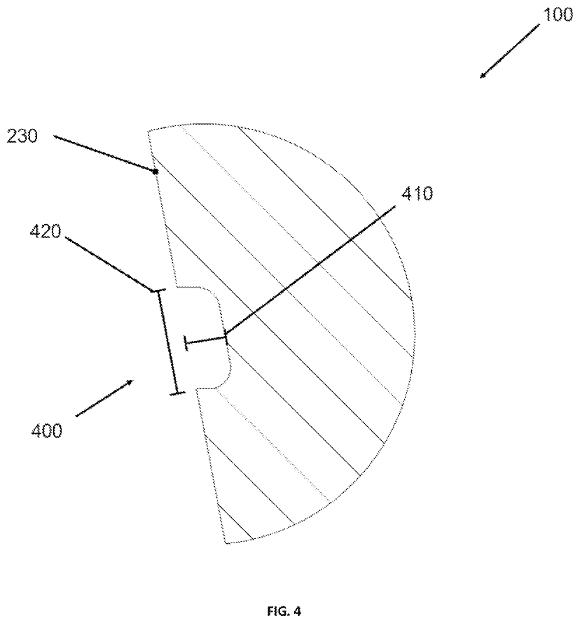

FIG. 4 an enlarged detail of the schematic longitudinal section from FIG. 4,

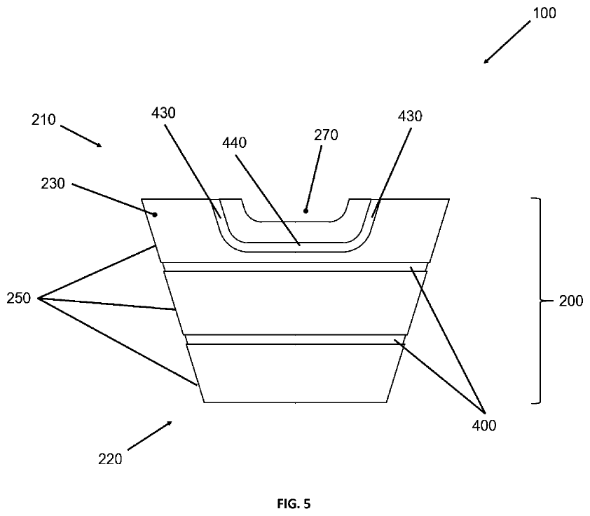

FIG. 5 a schematic side view of the augmentation device from FIGS. 1 and 2, with adapted cone size,

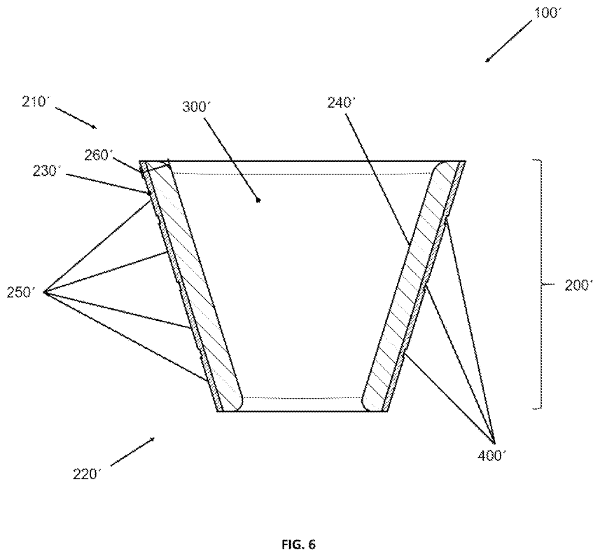

FIG. 6 a schematic longitudinal section of a further augmentation device, and

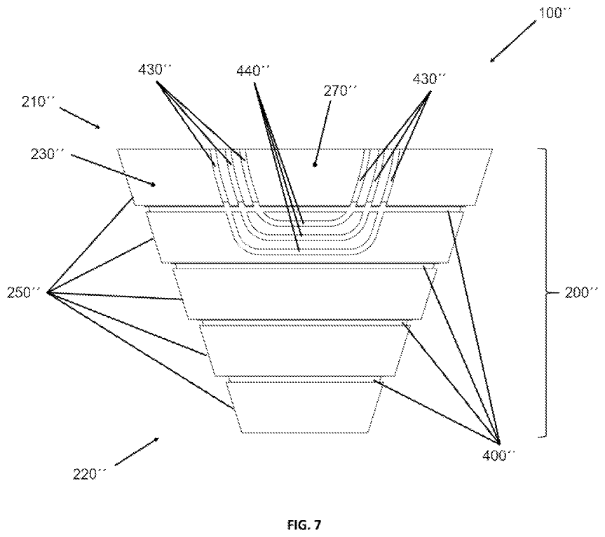

FIG. 7 a schematic side view of a further augmentation device with cone segments separated in a stepped manner.

### DETAILED DESCRIPTION

FIG. 1 shows a schematic side view of an exemplary embodiment of an augmentation device 100. The augmentation device 100 is formed as one piece and consists of a PMMA bone cement loaded with an antibiotic. The augmentation device 100 comprises a solid annular cone 200 comprising a proximal cone end 210 and a distal cone end 220, wherein a cone outer diameter, formed by a lateral surface 230 of the cone, decreases from the proximal cone end 210 in the direction of the distal cone end 220. In the shown embodiment, the cone outer diameter decreases substantially linearly from the proximal cone end 210 to the distal cone end 220.

A channel extends through the cone 200 from the proximal cone end 210 to the distal cone end 220.

Three grooves 400, which subdivide the cone into four annular cone segments 250, respectively extend radially in a complete circumference in the lateral surface 230 of the cone 200. The grooves 400 are thereby arranged in such a way that the cone segments 250 have a substantially equal axial extent. The grooves 400 serve as sawing guides so that one or more cone segments 250 can be separated from the augmentation device 100 by suitable means, for example with a saw, in a simplified and safe manner. A cone size of the augmentation device 100 is adapted with each separation of a single cone segment 250. For example, by separating a cone segment 250 at the proximal cone end 210, it occurs both that the axial extent of the cone 200 is reduced by substantially 25% and a maximum cone outer diameter of the cone 200 is reduced.

The grooves 400 respectively extend in a plane which extends perpendicular to a longitudinal axis 205 of the cone 200.

In the lateral surface 230, twelve axial grooves 430 continue to run. The axial grooves 430 extend over the cone segment 250 at the proximal cone end 210 and also partially over the cone segment 250 distally adjacent thereto. All axial grooves 440 are connected at least to the groove 400 at the proximal cone end 210, wherein four of the axial grooves 440 open into this groove 400, whereas the remaining axial grooves 400 extend beyond the groove 400 at the proximal cone end 210 until further in the direction of the distal cone end 220.

Two of the axial grooves 430 are connected to one another via a connecting groove 440 and form an axial groove pair. The axial grooves 440 form, with the groove 400 at the proximal cone end 210 and/or with the connecting grooves 440, conical parts 270 which can be simply and safely separated from the cone 200, for example by sawing, and allow an improved adaptation to the anatomical conditions of a patient.

FIG. 2 shows the augmentation device 100 from FIG. 1 in a side view, rotated by 90° about the longitudinal axis. It can be seen in FIG. 2 that the axial grooves 430 are arranged symmetrically so that conical parts 270 can be separated from the cone 200 on two sides at the proximal cone end 210.

FIG. 3 shows the augmentation device 100 from FIGS. 1 and 2 in a schematic longitudinal section. From FIG. 3 it is to be learned that the channel 300 is formed by a cone inner surface 240 and extends from the proximal cone end 210, through the cone 200, to the distal cone end 220. The cone 200 has a conical wall thickness 260 which is constant over the entire axial extent except for slight variances at the proximal cone end 210 and at the distal cone end 220. The cone wall thickness 260 of the shown embodiment of the augmentation device 100 is 10 mm. Furthermore, it can be seen that the cone 200 is of solid design, and therefore that there is no fluid-conducting connection through the cone 200 between the cone inner surface 240 and the lateral cone surface 230.

FIG. 4 shows an enlarged detail of the schematic longitudinal section of FIG. 3. FIG. 4 shows in particular an enlarged longitudinal section through one of the grooves 400 of the augmentation device 100. The groove 400 has a groove depth 410 extending perpendicular to the lateral surface 230 and a groove width 420 extending parallel to the lateral surface 230. In the shown embodiment of the augmentation device 100, the groove 40 has a groove depth 410 of 1 mm and a groove width 420 of 2.5 mm.

FIG. 5 shows the augmentation device 100 from FIGS. 1 to 4 in a schematic side view, with adapted cone size. In comparison to the preceding FIGS. 1 to 4, the cone segment 250 with the largest outer diameter was removed at the proximal cone end 210, so that the augmentation device 100 in FIG. 3 has a reduced maximum cone outer diameter. In addition, a conical part 270 has respectively been separated on both sides of the cone 200 along the middle pair of axial grooves 430, running on both cone sides, and their connecting groove 440.

FIG. 6 shows a schematic longitudinal section of a further exemplary augmentation device 100′. The embodiment of the augmentation device 100′ largely corresponds to the embodiments described above and shown in FIGS. 1 through 5, and therefore reference is made to the above description to avoid repetition. Modifications of any of the embodiments shown in FIGS. 1 to 5 have the same reference sign with an apostrophe.

The augmentation device 100′ differs from the augmentation device 100 from FIGS. 1 to 5 by a porous lateral surface 230′ formed from titanium. In the shown embodiment of the augmentation device 100′, the lateral surface 230′ is formed entirely of titanium.

FIG. 7 shows a schematic side view of another exemplary augmentation device 100″. The embodiment of the augmentation device 100″ largely corresponds to the embodiments described above and shown in FIGS. 1 to 5 and FIG. 6, and therefore reference is made to the above description to avoid repetition. Modifications to an embodiment shown in FIGS. 1 to 5 and FIG. 6 have the same reference sign with two apostrophes.

The augmentation device 100″ differs from the augmentation devices 100 and 100′ from FIGS. 1 to 5 and FIG. 6 by cone segments 250″ that are recessed from one another in a stepped manner. Due to the step-like cone segments 250″, the cone outer diameter of the augmentation device 100″ does not decrease substantially linearly from the proximal cone end 210″ to the distal cone end 220″, but rather “abruptly” between the cone segments 250″.

### REFERENCE NUMERALS

100, 100′, 100″ Augmentation device
200, 200′, 200″ Cone
205 Longitudinal axis of the cone
210, 210′, 210″ Proximal cone end
220, 220′, 220″ Distal cone end
230, 230′, 230″ Lateral cone surface
240, 240′ Cone inner surface
250, 250′, 250″ Cone segment
260, 260′ Cone wall thickness
270, 270″ Conical part
300, 300′ Channel
400, 400′, 400″ Groove
410 Groove depth
420 Groove width
430, 430″ Axial groove
440, 440″ Connecting groove

What is claimed is:

1. An augmentation device comprising
an annular cone surrounding a channel which extends from a proximal cone end to a distal cone end of the cone through said cone,
wherein
the cone consists by at least 50 percent by volume, based on the total volume of the cone, of a biocompatible polymer, and is subdivided, by at least three radially circumferential grooves in a cone lateral surface opposite the channel, into annular cone segments,
wherein the grooves have a groove depth of at least 1 mm and a groove width of at least 1 mm, and form sawing guides in order to adapt a cone size of the cone by separating one or more cone segments.

2. The augmentation device according to claim 1, wherein the lateral cone surface is roughened or porous at least in portions.

3. The augmentation device according to claim 1, wherein the lateral cone surface is formed by at least 70 percent by area, based on the entire lateral cone surface, of tantalum, a tantalum alloy, titanium, a titanium alloy, or stainless steel.

4. The augmentation device according to claim 1, wherein the biocompatible polymer is a PMMA bone cement.

5. The augmentation device according to claim 4, wherein the PMMA bone cement contains at least one antibiotic.

6. The augmentation device according to claim 1, wherein the cone comprises radially circumferential inner grooves in a cone inner surface facing toward the channel, which inner grooves are arranged opposite the grooves.

7. The augmentation device according to claim 1, wherein the lateral cone surface comprises at least two axially extending axial grooves having an axial groove depth of at least 1 mm and an axial groove width of at least 1 mm, each of which is connected at least to one of the grooves.

8. The augmentation device according to claim 1, wherein the cone segments are separated from one another in a stepped manner.

9. A method for adapting a cone size of an augmentation device according to claim 1, comprising a step of separating one or more cone segments at the proximal cone end and/or at the distal cone end by sawing, cutting, or breaking along the radially circumferential groove or grooves.

10. The method according to claim 9 for adapting an augmentation device comprising:
an annular cone surrounding a channel which extends from a proximal cone end to a distal cone end of the cone through said cone,
wherein
the cone consists by at least 50 percent by volume, based on the total volume of the cone, of a biocompatible polymer, and is subdivided, by at least three radially circumferential grooves in a cone lateral surface opposite the channel, into annular cone segments,
wherein the grooves have a groove depth of at least 1 mm and a groove width of at least 1 mm, and form sawing guides in order to adapt a cone size of the cone by separating one or more cone segments,
comprising a step of separating a conical part from the cone by sawing, cutting, or breaking along two of the axially extending axial grooves.
